(19) United States
(12) Patent Application Publication
(10) Pub. No.: US 2019/0336040 A1
(43) Pub. Date: Nov. 7, 2019

(54) WALKING ASSIST DEVICE

(71) Applicant: Chang Gung University, Taoyuan City (TW)

(72) Inventors: YA-JU CHANG, Taoyuan City (TW); JIUNN-WOEI LIAW, Taoyuan City (TW); HSIAO-LUNG CHAN, Taipei City (TW)

(21) Appl. No.: 16/515,022

(22) Filed: Jul. 17, 2019

Related U.S. Application Data
(63) Continuation-in-part of application No. 15/669,318, filed on Aug. 4, 2017.

(30) Foreign Application Priority Data
Aug. 5, 2016 (TW) 105124907
Jul. 17, 2018 (TW) 107124685

(51) Int. Cl. A61B 5/103; A61B 5/11; A61B 5/00

(52) U.S. Cl. CPC A61B 5/1038; A61B 5/112; A61B 5/486; A61B 2505/09; A61B 5/6807; A61B 2562/0219; A61B 2562/0247; A61B 5/742

(57) ABSTRACT

The present disclosure relates a walking assist device, including: a sensor configured to detect a movement of an user and to generate detection signals, a step length module configured to calculate a step length, a controller connected to the sensor and the step length module, and an output module. The controller is configured to determine a step status according to the detection signals, and to generate control signals according to the step status and the step length. The output module is disposed on a shoe body. The output module connects to the controller, and the output module is configured to issue a step cue according to the control signals.

### TECHNICAL FIELD

The present disclosure relates to a walking assist device, more particularly to a walking assist device for assisting users in gait rehabilitation and step training.

### DESCRIPTION OF THE RELATED ART

There are many diseases, such as stroke, Parkinson's disease, cerebral palsy, and so on, which may cause a problem of “gait difficulty” and may greatly reduce the living-ability and the quality of life. “Gait training” is an important process for the patients having the gait difficulty to regain the independent walking ability. Currently, the gait training in clinical mostly relies on the therapists to supervise and to subjectively judge the patient's ability and to give corresponding external cues to induce the correct gait. However, when the therapist is not supervising, the patients may walk in a wrong posture and may develop a wrong gait pattern due to the absence of the cues, which are not facilitated to further gait rehabilitation.

A conventional visual cuing device for gait difficulty is to install a laser light emitter on patient's crutches, and laser beams are emitted to the ground to guide the patient to step forward. However, when there is only one laser light, the patient may only able to guide the first step, the other foot of the patient may not able to step further to the correct distance, and the patient cannot walk in a normal reciprocal pattern. In addition, the patient with gait difficulty (e.g. gait freezing) may not always need to use crutches. Therefore, when gait difficulty (e.g. gait freezing) occurs and the crutch with the laser light emitter is not around, the patient may not be able to make correct striding actions.

Moreover, the conventional step cuing device were usually placed on external equipment, such as walking assist devices, crutches, wheelchairs, and so on. For patients who do not need to use such walking assistive devices, their gait pattern might be worsen by those walking assistive devices.

Therefore, it is necessary to provide a cuing device capable of guiding the patients having the gait difficulties to walk without using the external equipment.

### SUMMARY

In one aspect, the present disclosure relates to a walking assist device, including: a sensor being configured to detect a movement of an user and to generate detection signals; a step length module being configured to calculate a step length; a controller being connected to the sensor and the step length module, the controller is configured to determine a step status according to the detection signals, and to generate control signals according to the step status and the step length; and an output module being disposed on a shoe body, the output module connects to the controller, and the output module is configured to issue a step cue according to the control signals.

The sensor includes a tri-axial accelerometer, and the detection signals include X-axis acceleration signals, Y-axis acceleration signals, and Z-axis acceleration signals; the controller is configured to determine the step status according to the Z-axis acceleration signals, and the step length module is configured to determine the step length according to the X-axis acceleration signals and the Y-axis acceleration signals.

The step length module is configured to compensate the X-axis acceleration signals and the Y-axis acceleration signals according to the Z-axis acceleration signals.

The sensor including a plurality of detection units is disposed on a bottom of the shoe body, and each of the detection units is configured to detect a pressure generated by a foot on the bottom of the shoe body. In addition, the detection units are configured on a front detection area, an inner side detection area, an outer side detection area, and a rear detection area.

The above summary of the present disclosure is to provide a basic description of the various aspects and features of the present disclosure. It is not a detailed description. Its purpose is not to specifically recite keys or critical elements of the present disclosure, and it is not intended to limit the scope of the present disclosure. It merely presents a few concepts of the present disclosure in a concise manner.

### BRIEF DESCRIPTION OF THE DRAWINGS

FIG. 1 is a diagram illustrating a walking assist device in accordance with one embodiment of the present disclosure.

FIG. 2 is a diagram illustrating a sensor of the walking assist device in accordance with one embodiment of the present disclosure.

FIG. 3 is a diagram illustrating examples of step cues in accordance with one embodiment of the present disclosure.

FIG. 4 is a diagram illustrating a user who performs a complete cycle of step status.

FIG. 5a is diagram illustrating the walking assist device assisting the user to perform a move-straight forward motion in accordance with one embodiment of the present disclosure.

FIG. 5b is diagram illustrating the walking assist device assisting the user to perform a turn-left motion in accordance with one embodiment of the present disclosure.

FIG. 5c is diagram illustrating the walking assist device assisting the user to perform a turn-right motion in accordance with one embodiment of the present disclosure.

FIG. 6 is a diagram illustrating a walking assist device in accordance with another embodiment of the present disclosure.

FIG. 7 is a diagram illustrating position of emitting units in accordance with one embodiment of the present disclosure.

FIG. 8 is a diagram illustrating the emitting units of the walking assist device in accordance with another embodiment of the present disclosure.

### DESCRIPTION OF THE PREFERRED EMBODIMENTS

To clarify the purpose, technical solutions, and the advantages of the disclosure, embodiments of the present disclosure will now be described more fully hereinafter with reference to the accompanying drawings.

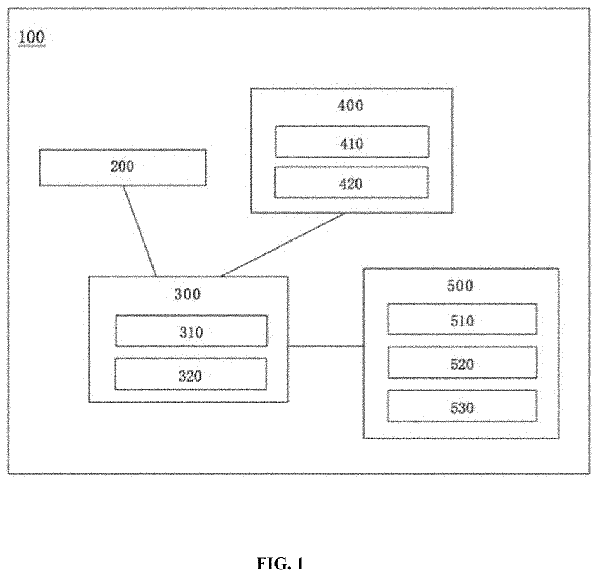

Referring to FIG. 1, FIG. 1 is diagram illustrating a walking assist device in accordance with one embodiment of the present disclosure, the walking assist device 100 is configured to output a step cue to assist a user when the user is walking. The walking assist device 100 includes a sensor 200, a controller 300, a step length module 400, and an output module 500.

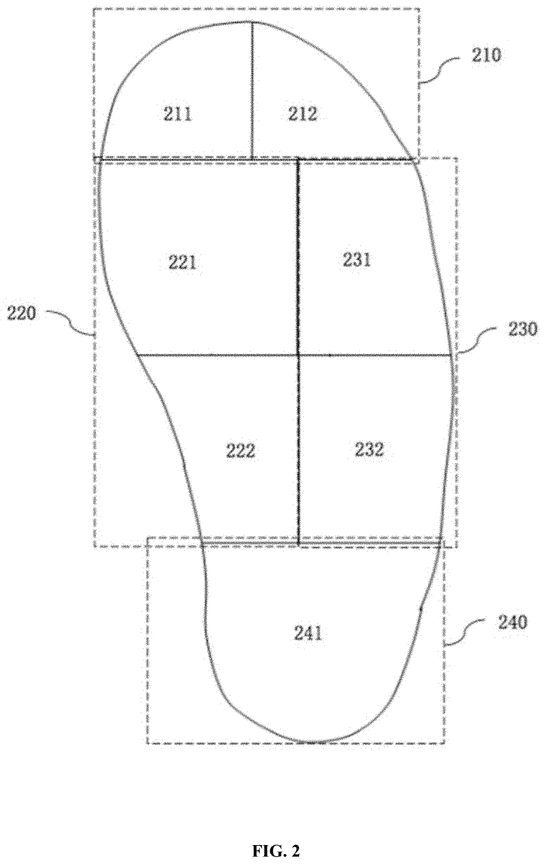

Referring to FIG. 2, the sensor 200 includes a forefoot detection area 210, a detection area of an inner side of the foot 220, a detection area of an outer side foot 230, and a rear foot detection area 240. The sensor 200 further includes a plurality of detection units 211, 212, 221, 222, 231, 232, and 241. Each of the detection units 211, 212, 221, 222, 231, 232, and 241 is configured to detect a pressure on a bottom of a shoe body generated by the user's foot.

In one example, the plurality of detection units 211, 212, 221, 222, 231, 232, and 241 may be pressure sensors. The plurality of detection units are configured in an insole to form a pressure pad, and the pressure pad is disposed inside the shoe body.

In one example, the sensor 200 may be a gyroscope or an accelerometer, which may not be disposed on the insole.

The step length module 400 may include a step length calculating unit 410 and a step length outputting signal module 420. The step length calculating unit 410 is configured to calculate a step length of one leg of the user. The step length outputting signal unit 420 is configured to transmit step length signals to the output module 500 or to the controller 300 to directly or indirectly control an outputted distance of the step cue.

In one example, the step length may be calculated by detecting an acceleration situation or a turning situation of the user when the user is moving via the gyroscope, the accelerometer, or a magnetometer. According to a speed variation of user's bodies during the movement, the step length, which is more suitable for the user's own step length, cadence, and step velocity may be calculated. The accelerometer may adopt a space accelerator, a uniaxial accelerator, a biaxial accelerator, or a tri-axial accelerator.

The controller 300 connects to the sensor 200. The controller 300 includes a status determining unit 310 and a status signal outputting unit 320. The status determining unit 310 is configured to determine a step status of the user by a predetermined algorithm according to a pressure change amount generated on the bottom of the shoe body by the user's foot and/or an acceleration change amount of the foot detected by the sensor 200. The status determining unit 310 is configured to output step status signals to the output module 500 by the status signal outputting unit 320. Moreover, the controller 300, which is configured on right and left feet, may transmit or/and receive relevant information via a wireless communication.

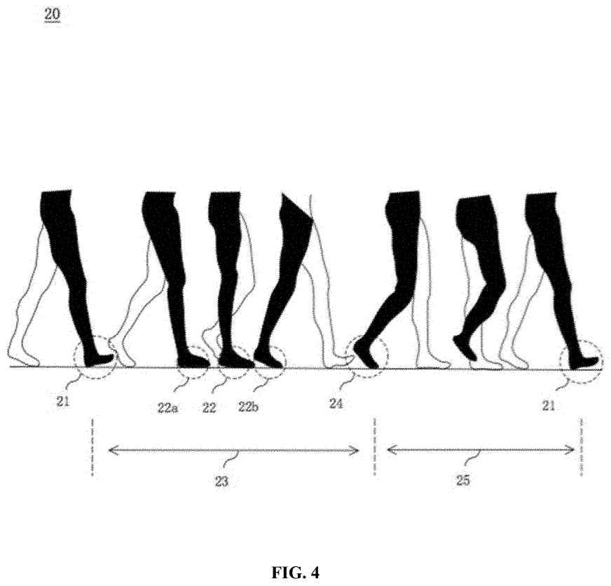

Referring to FIG. 4, in one example, the phase status of a gait cycle, when the user is moving, may include a stand phase 23 and a swing phase 25. The stand phase 23 may include a heel-strike status 21, a mid-stance status 22, and a toe-off status (a push-off status) 24. A period of the mid-stance status 22 is from a status that the foot lies in flat 22a to a status that a heel is off a ground 22b.

The output module 500 includes a signal receiving unit 510, an emitting unit 520, and an adjusting module 530. In one example, the output module 500 connects to the controller 300 and the step length module 400. The signal receiving unit 510 is configured to drive the emitting unit 520 of the output module 500 to issue the step cue suitable for the user to a front of the shoe body according to the step length signals transmitted by the step length module 400 and the step status signals transmitted from the controller 300. As such, the user may be able to confirm a position of the step via the step cue, and to keep moving. In another example, the controller 300 is configured to receive the step length signals of the step length module 400, and to generate control signals to the output module 500 according to the step length signals and the step status signals. The output module 500 is configured to issue the step cue according to the control signals.

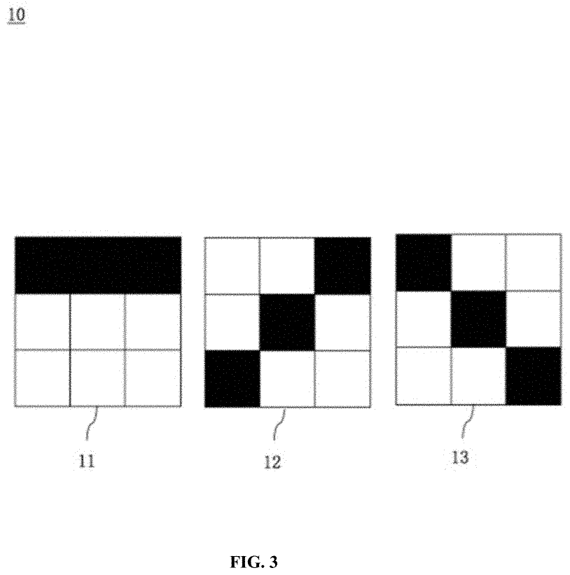

The step cue generated by the emitting unit 520 may have a variety of examples. In one example, as shown in FIG. 3, the step cue issued by the emitting unit 520 may be a light of a multi-grid array of 3×3, 4×4, 3×4 or images. The light of M×N multi-grid array or images may form the step cue indicating a move-straight forward motion 11, a turn-left motion 12, and a turn-right motion 13 for a short-distance, a medium-distance, or a long-distance to guide the user to move-straight forward, turn-left, or turn-right. The step cue indicating the move-straight forward motion 11 shown in FIG. 3 is the step cue of the long-distance.

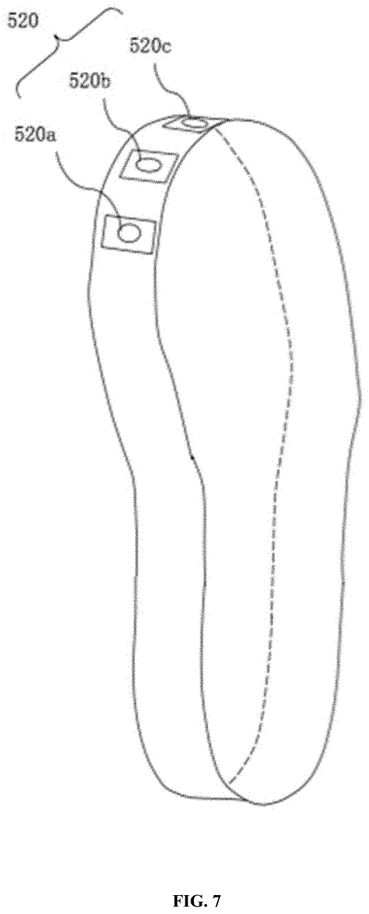

In another example, the emitting unit 520 is configured to issue the step cues indicating the move-straight forward motion for the long-distance, the move-straight forward motion for the short distance, and a turning motion. Referring to FIG. 7, it is noted that a position and a number of the emitting unit 520 are illustrative, and the present disclosure is not limited thereto. The output module 500 may include three emitting units 520. A first emitting unit 520a is configured on an inner side of the foot on a front portion of the shoe body. A second emitting unit 520b is configured on a front inner side of the foot. A third emitting unit 520c is configured on a front of the foot. The first emitting unit 520a and the third emitting unit 520c are respectively configured to issue a straight-line or the images for the long-distance and for the long-distance, i.e., the step cue 10. The second emitting unit 520b is configured to issue the step cue 10 to guide the user to turn. The three emitting units 520a, 520b, and 520c are configured to issue the step cue 11 indicating the move-straight forward motion for the short-distance (by the first emitting unit 520a), the step cue 11 indicating the move-straight forward motion for the long-distance (by the third emitting unit 520c), the step cue 12 indicating the turn-left motion (by the second emitting unit 520b configured on the left foot), and the step cue 13 indicating the turn-right motion (by the second emitting unit 520b configured on the right foot). As such, deviations occurred in oblique light or images when the user is turning may be avoided. For example, the users having the stroke may only require the step cue for single-foot, or require two different step cues for two feet (one foot is for long-distance and the other foot is for short-distance). The output module 500 having the three emitting units 520 may be able to adjust accordingly, as so to satisfy the user's demand.

The adjustment unit 530 is configured to adjust a brightness or a color of the light or images outputted by the light emitting unit according to a brightness of environment. The adjustment unit 530 is further configured to adjust an output frequency of the light emitting unit 520 according to the user's demand. In another example, the adjustment unit 530 is configured to turn off the emitting unit or to issue the step cue for only one side in response to the disease or the user's demand.

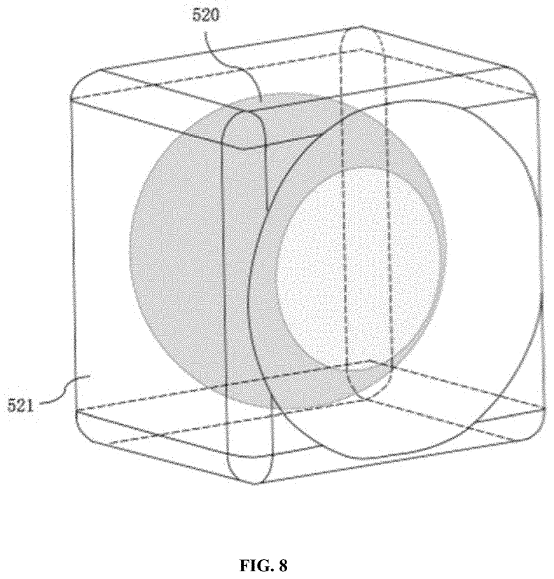

In one example, the emitting unit 520 may be a light-emitting diode (LED), a laser emitter, an image emitter, a LED group, and a laser emitter group. But the present disclosure is not limited thereto. Referring to FIG. 8, the emitting unit 520 may be spherical, and may be configured in a shell 521. The emitting unit 520 may rotate in 360 degrees within the shell 521, and is configured to issue the step cue, which may be the light or the images. As such, an emitting range of the step cue may become wider. In another example, at least one circular hole (not shown) may be configured around the shell 521 to provide the emitting unit 520 an even wider emitting range for issuing the step cue.

The walking assist device may further include a power supply unit (not shown) configured on the shoe body. The power supply unit respectively connects the sensor 200, the controller 300, the step length module 400, and the output module 500. In one example, the power supply unit may be a secondary battery or a primary battery, but the present disclosure is not limited thereto. Any power supply unit capable of supplying power may be adopted in the present disclosure.

The controller 300, the step length module 400, the output module 500, and the power supply unit (not shown) may be disposed on the shoe body by any of the known means. In one example, an air cushion at the bottom of the shoe body is configured with an accommodating space for installing or removing the step length module, the controller, the output module, and the power supply unit.

The algorithm for determining the step status of the user when the user is moving according to the sensor 200 is described in below.

In one example, the status determining unit 310 of the controller 300 is configured to determine the user's step status by an internal algorithm via the user's movement detected by the sensor 200. The status determining unit 310 is configured to output the control signals to the output module 500 via the status signal outputting unit 320 according to the step status signals. As such, a projecting module of the emitting unit 520 may be driven to issue the step cue to the front of the shoe body at a right timing, so as to guide the user properly.

Referring to FIG. 2, FIG. 3, and FIG. 4, taking the left foot as an example, when pressure components are detected within the forefoot detection area 210, the rear foot detection area 240, the detection area of the inner side of the foot 220, and the detection area of the outer side foot 230 (i.e., the detection units 211, 212, 221, 222, 231, 232, 241 shown in FIG. 2), the status determining unit 310 is configured to determine the step status 20 to be at the stand phase 23. When no obvious pressure is detected within the plurality of detection area, the status determining unit 310 is configured to determine the step status 20 to be at the swinging phase 25. When the obvious pressure is only detected within the rear foot detection area 240 (i.e., the detection unit 241 shown in FIG. 2), the status determining unit 310 is configured to determine the step status 20 to be at the heel-strike status 21. When the obvious pressure is detected within at least two of the forefoot detection area 210, the rear foot detection area 240, the detection area of the inner side of the foot 220, and the detection area of the outer side foot 230 (i.e., the detection units 211, 212, 221, 222, 231, 232, 241 shown in FIG. 2), the status determining unit 310 is configured to determine the step status 20 to be at the mid-stance status 22. When no obvious pressure is detected within the rear foot detection area 240 (i.e., the detection unit 241 shown in FIG. 2) and the pressure detected within the forefoot detection area 210 decreases to no obvious pressure, the status determining unit 310 is configured to determine the step status 20 to be at the toe-off status 24. Regarding “obvious or non-obvious”, it can be compared with at least one critical value. When a pressure value is greater than the at least one critical value, it is determined to be obvious. When the pressure value is less than the at least one critical value, it is determined to be non-obvious.

Taking the left foot as an example, when the obvious pressure is detected within the forefoot detection area 210 (i.e., the detection units 211 and 212 shown in FIG. 2) and a pressure increase amount detected within the detection area of the outer side foot 230 (i.e., the detection units 231 and 232 shown in FIG. 2) is similar to a pressure increase amount detected within the detection area of the inner side of the foot 220 (i.e., the detection units 221 and 222 shown in FIG. 2), the status determining unit 310 is configured to determine the step status 20 to be at the mid-stance status 22 of the move-straight forward motion 11. When the obvious pressure of the user is detected within the forefoot detection area 210 (i.e., the detection units 211 and 212 shown in FIG. 2) and a pressure increase amount detected within the detection area of the outer side foot 230 (i.e., the detection units 231 and 232 shown in FIG. 2) is greater than the pressure increase amount detected within the detection area of the inner side of the foot 220 (i.e., the detection units 221 and 222 shown in FIG. 2), the status determining unit 310 is configured to determine the step status 20 to be the mid-stance status 22 of the turn-left motion 12. Similarly, taking the right foot as an example, the status determining unit 30 is configured to determine the step status 20 to be at the mid-stance status 22. The mid-stance status 22 may include the move-straight forward motion and the turn-right motion according to a slight difference of the pressure detected within the plurality of the detection area. When the obvious pressure of the user is detected within the forefoot detection area 210 (i.e., the detection units 211 and 212 shown in FIG. 2) and the pressure increase amount detected within the detection area of the outer side foot 230 (i.e., the detection units 231 and 232 shown in FIG. 2) is greater than the pressure increase amount detected within the detection area of the inner side of the foot 220 (i.e., the detection units 221 and 222 shown in FIG. 2), the status determining unit 310 is configured to determine the step status to be at the mid-stance status 22 of the turn-right motion 13.

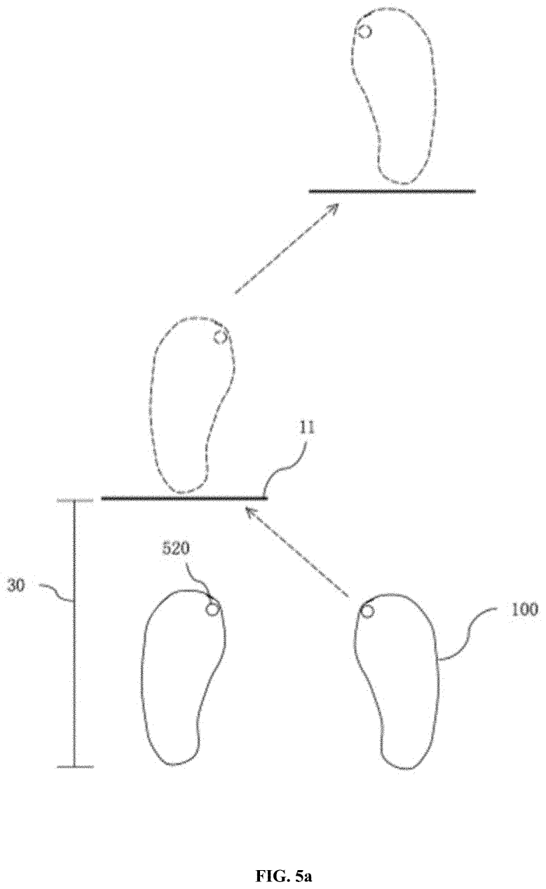

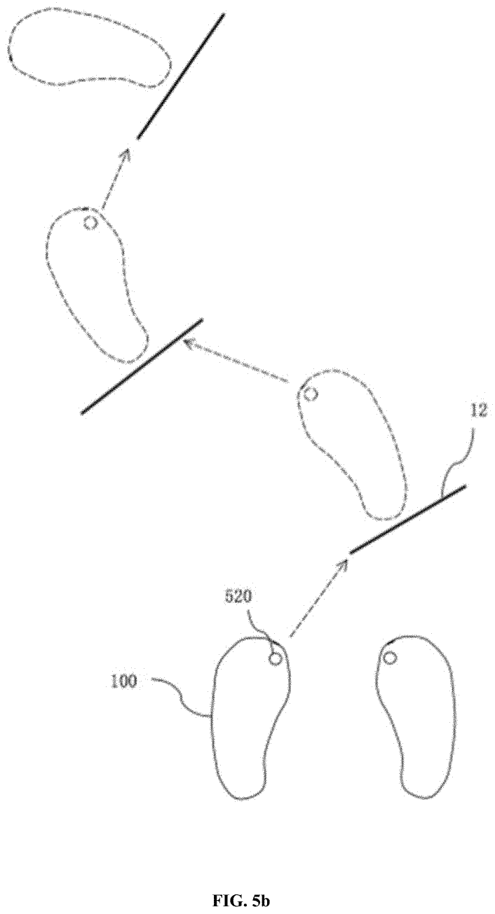

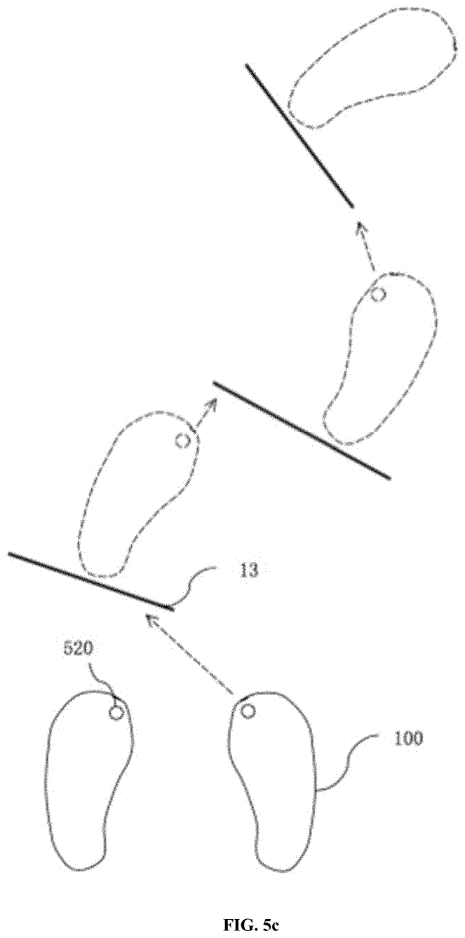

Referring to FIG. 4, FIG. 5a, FIG. 5b, and FIG. 5c, FIG. 5a is a diagram illustrates the walking assist device 100 assisting the user to perform the move-straight forward motion. When the user is moving and the step status of the right foot is at the mid-stance status 22, the step cue 11 is issued by the emitting unit 520 of the right foot to guide the left foot to step forward. When the step status of the left foot is at the mid-stance status 22, the step cue 11 is issued by the emitting unit 520 of the left foot to guide the right foot to step forward. As such, the user may be able to move forward along the step cues.

When the user is about to turn left, the step cue 12 indicating the turn-left motion may be issued by the left foot, which is at the mid-stance status 22, to guide the right foot to step to left front. When the right foot has stepped to left front, the right foot is configured to be at the mid-stance status 22. Then, the step cue 11 indicating the move-straight motion is issued by the right foot to guide the left foot to step forward. As such, the user may be able to turn left along the step cue.

When the user is about to turn right, the step cue 13 indicating the turn-right motion may be issued by the right foot, which is at the mid-stance status 22, to guide the left foot to step to right front. When the left foot has stepped to right front, the left foot is configured to be at the mid-stance status 22. Then, the step cue 11 indicating the move-straight forward motion is issued by the left foot to guide the right foot to step forward. As such, the user may be able to turn right along the step cue. The patients having the gait difficulty may maintain a balance when moving by a guidance of the above left and right inconsistent step cues.

The step cue 11 indicating the move-straight forward motion, the step cue 12 indicating the turn-left motion, and the step cue 13 indicating the turn-right motion may be issued by the single-foot when the single-foot is on the ground (at the stand phase). As such, a deviation resulting from a moving of the foot may be avoided.

The walking assist device 100 is configured to guide the user for a turning radius of rotation by the outputted distance of the step cue issued by the emitting unit 520. When the emitting unit 520 issues the step cue 11 indicating the move-straight forward motion for the long-distance, the turning radius of the user may become greater. When the emitting unit 520 issues the step cue 11 indicating the move-straight forward motion for the short-distance, the turning radius of the user may become less.

The status determining unit 310 and the algorithm may be implemented by a programmable logic device, a logic circuit, or a processor/controller with a firmware or software.

When two feet of the user are at the mid-stance status 22 in a long-term and a pressure value detected within the at least one of the fore foot detection area 210 and the rear foot detection area 240 of the at least one foot is determined to shake (tremor) obviously, i.e., a shake amount is greater than a shake threshold, the status determining unit 310 is configured to determine the user has a striding intend and the user is at a gaiting freeze status. The status signal outputting unit 320 is configured to transmit signals of the step status 20 to the output module 500 of the foot having less shake amount, which has no striding intend, to drive the output module 500 to issue the step cue 10 to the front of the shoe body. As such, a striding position may be provided to the patient having Parkinson's disease to guide the foot having greater shake amount (which has the striding intend) to step forward. Moreover, when the status determining unit 310 determines that the two feet of the user are at the mid-stance status 22 and the pressure value detected within the forefoot detection area 210 and the rear foot detection area 240 of the left and right feet has no obvious shake (i.e., the shake amount is less than the shake threshold), the status determining unit 310 is configured to determine that the user is at a standing status, and is configured to disable the output module 500 to reduce power consumption.

The detail that the step length calculating unit 410 of the step module 400 determines the step length when the user is moving is described in below.

Referring to FIG. 4, the step length is configured to be calculated by a time interval of the single-foot moving from the swing phase 25 (i.e., the toe-off status 24) to the heel strike status 21. The step length is referred to as the time interval of the swing phase, and is configured to be as the time interval for the single-foot movement. The time interval of the swing phase may be obtained by output signals of the corresponding detection unit of the sensor 200. The step length calculating unit 410 is configured to calculate the step length of one single leg of the user according to the time interval of the swing phase. The step length calculating unit 410 is configured to calculate the step length in the following manners.

In one example, obtaining a variation of an acceleration component in an

X-axis and a Y-axis during the time interval of the swing phase by a dual-axis accelerometer, calculating (synthesizing) an acceleration curve on an X-Y plane by the acceleration components in the X-axis and Y-axis, obtaining a forward speed by performing an integral calculation on the acceleration curve, and obtaining the step length by performing the integral calculation on the forward speed.

In another example, obtaining a variation of the acceleration component in the X-axis, the Y-axis, and a Z-axis during the time interval of the swing phase by a tri-axial accelerometer. Since the Z-axis of the accelerometer is not always maintained to be on the Z-axis, and an angle (inclination angle) formed by the Z-axis of the accelerometer and a gravitational acceleration “g” (which is a constant value of 9.8) is obtained by the Z-axis acceleration of the accelerometer and the gravitational acceleration “g”, so as to compensate the X-axis acceleration signals and the Y-axis acceleration signals. The forward speed may be obtained by performing the integral calculation on the acceleration curve on the X-Y plane obtained (synthesized) by the compensated X-axis acceleration component and the Y-axis acceleration component. The step length may be obtained by performing the integral calculation on the forward speed. The sensor 200 of the present disclosure may adopt the Z-axis acceleration as a criterion for detecting the step status of the user. In other words, only one tri-axial accelerometer is adopted. Z-axis acceleration data is adopted as a criterion for determining the step status of the user, and X-axis acceleration data and Y-axis acceleration data are adopted to calculate the step length of the user.

In another example, an infrared emitter is disposed on the inner side of one the shoe body, and two infrared receivers are respectively disposed on the inner side of the front portion and the inner side of a rear portion of the other shoe body. A relative distance between the two infrared receivers is configured to be as “D”. The relative distance “D” is divided by a time difference between the infrared ray received by the two receivers, and the forward speed may be obtained. The step length may be estimated by multiplying the forward speed by the time interval of the swing phase.

The following description is a detail of adjusting the outputted distance of the step cue 10 issued by the output module 500 by adopting a variety of feedback control methods.

In one example, adjusting the step length depending on whether the step length matches the outputted distance or not. A result of comparing the step length calculated by the step length module 400 and the outputted distance of the step cue 10 issued by the output module 500 (for example, determining whether the calculated step length is greater than the outputted distance of the step cue 10) is transmitted to the controller 300 to adjust the outputted distance of the step cue 10 issued by the output module 500.

In another example, when the controller 300 determines the user has a gait variation feature, the outputted distance of the next step cue 10 may require to be adjusted (for example, be reduced) according to the gait variation feature. The gait variation feature may include a pressure variability between each step (left and right feet) during the stand phase, a center distance variability of a single-foot, a ratio of the stand phase to the swing phase between each step (left and right feet), a ratio variability of the stand phase to the swing phase of each step, a pressure variation ratio of the left foot to the right foot, or a ratio variation of the stand phase to the swing phase with respect to the left foot and the right foot. For example, when the step length of the user is greater than the step cue 10, the above gait variation features may appear. The above gait variation features may be regarded as a poor gait of the user and may be configured to be unstable. The outputted distance is required to be adjusted (reduced) in the next step cue 10 to prevent the user from falling down. If the sensor 200 adopts a plurality of detection units, the controller 300 may obtain all of the gait variation features according to a previous step status and a current step status. If the sensor 200 is the accelerometer, the controller 300 may obtain the ratio of the stand phase to the swing phase between each step (left and right feet), the ratio variability of the stand phase to the swing phase of each step, and a ratio variability of the stand phase to the swing phase with respect to the same foot according to the previous step status and the current step status. In another example, if the sensor 200 adopts the plurality of detection units, the pressure variability of the single-foot during the stand phase and the center distance variability of the single-foot may be calculated by the pressure variation detected within the detection area of the inner side of the foot 220 and the detection area of the outer side foot 230.

In one example, the step cue 10 may be issued from the corresponding foot, that is, when the left foot issues the step cue 10, the right foot is configured to move to the position of the step cue 10. When the right foot issues the step cue 10, the left foot is configured to move to the position of the step cue 10.

In another example, the present disclosure may further include a memory storage unit (not shown) connecting to the output module 500. When the user has completed a complete left and right feet striding cycle by using the walking assist device, the memory storage unit is configured to store the time interval of each step status and the step length that the user is accustomed to, thereby relevant parameters (for example, the outputted distance or an emitting angle of the step cue 10) of the output module 500 may be adjusted after an analysis, so as to optimize the step cue 10 for the user.

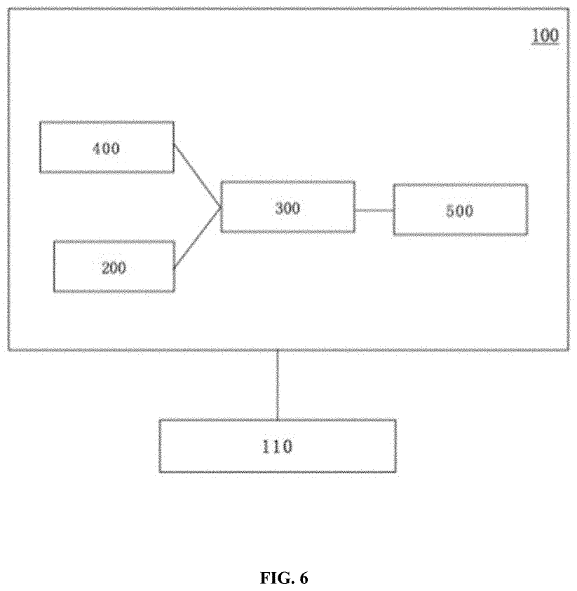

Referring to FIG. 6, the walking assist device 100 of the present disclosure may be wirelessly connected to a control device 110. The control device 110 may include three control modes: a move-straight forward mode, a turn-left mode, and a turn-right mode. The user may control a moving direction according to the demands. For example, the user selects the turn-right mode, the control device 110 may wirelessly transmit signals indicating the turn-right motion to the output module 500, and the step cue 10 indicating the turn-right motion may be issued by the emitting unit 520. As such, the user may be guided to turn right. The wireless connection between the control device 110 and the output module 500 may be a Bluetooth connection, a wireless network connection, or an infrared connection. In another example, the controller 300 of the present disclosure may further include a voice control function. Such that, the controller 300 is configured to control and adjust the output module 500 by distinguishing a voice input (for example, “move-straight forward”, “turn left”, “turn right”, and “freeze”) of the user.

The walking assist device 100 of the present disclosure may further connect to an external mobile device (not shown). The external mobile device is configured to receive the pressure variation, the step status 20, the step length 30, and the step cue 10 of the user detected by the walking assist device 100, and to output information to a display of the mobile device. As such, the user may clearly understand a striding record, and then adjust and correct the posture after. The mobile device may further include a detection mechanism configured to detect whether the user successfully steps along the step cue 10. If the user successfully steps along the step cue 10, the mobile device sends reward signals (for example, a ringing tone) to encourage the user to perform a correct striding practice. If the user does not successfully step along the step cue 10, the mobile device sends cue signals (for example, a warning tone or vibration) to guide the user that the step is not correct.

In view of the above, the sensor, the controller, the step length module, and the output module of the walking assist device of the present disclosure are cooperative with each other to provide a training, which is cooperating with the step cue, similar to a daily walk to the user. The walking assist device is further capable of determining the step status of the user and the step length that the user is accustomed to, and adjusting and outputting the step cue suitable for the user. As such, the patient having the gait difficulties may be assisted to regain the walking ability, and, for training athletes, the step length may be improved.

The above description is merely the embodiments in the present disclosure, the claim is not limited to the description thereby. The equivalent structure or changing of the process of the content of the description and the figures, or to implement to other technical field directly or indirectly should be included in the claim. All other embodiments obtained by a person of ordinary skill in the art based on the embodiments of the present disclosure without creative efforts shall fall within the protection scope of the present disclosure.

What is claimed is:

1. A walking assist device, comprising:
a sensor module configured to generate a detected signal corresponding to a movement of a user;
a step length module configured to calculate a step length corresponding to the movement;
a controller coupled to the sensor module and the step length module, the controller is configured to determine a step status by the detected signal, and generate a control signal by the step status and the step length; and
an output module, coupled to the controller, the output module is configured to output a step cue by the control signal.

2. The walking assist device of claim 1, wherein the detected signal comprises an X-axis acceleration signal, a Y-axis acceleration signal, and a Z-axis acceleration signal; wherein the controller determines the step status by the Z-axis acceleration signal, and the step length module calculates the step length according to the X-axis acceleration signal and the Y-axis acceleration signal.

3. The walking assist device of claim 2, wherein the step length is compensated according to the Z-axis acceleration signal.

4. The walking assist device of claim 1, the sensor module comprises a plurality of detection units, and each of the detection units detects a pressure generated by a foot on the bottom of the shoe body; and the detection units are configured on a front detection area, an inner side detection area, an outer side detection area, and a rear detection area.

5. The walking assist device of claim 4, wherein at least one of a pressure variability and a center of pressure (COP) variability during a single-foot-stand phase is obtained by a pressure of the inner side detection area and the outer side detection area.

6. The walking assist device of claim 4, wherein the controller is configured to determine one of a move-straight forward motion and a turning motion by a pressure increase amount detected within the inner side detection area and the outer side detection area.

7. The walking assist device of claim 1, wherein the controller calculates a forward speed, to obtain a time interval of a swing phase by the detection signal, and to obtain the step length by the forward speed and the time interval of the swing phase.

8. The walking assist device of claim 1, wherein the controller is configured to determine a gait variation feature according to the detection signal, and to adjust an outputted distance of the step cue issued by the output module according to the gait variation feature.

9. The walking assist device of claim 8, wherein the gait variation feature comprises at least one of a pressure variability during the single-foot-stand phase, the center of pressure (COP) variability during the single-foot-stand phase, a ratio of a stand phase to a swing phase between each step, a pressure variation ratio of a left foot to a right foot, or a ratio variation of the stand phase to the swing phase with respect to the left foot and the right foot.

10. The walking assist device of claim 1, wherein the controller is configured to adjust the outputted distance of the step cue issued by the output module according to the previous step length calculated by the step length module and the previous step length issued by the output module.

11. The walking assist device of claim 1, wherein the controller is configured to compare a shake amount of the detection signal with a shake threshold to determine one of a gaiting freeze status and a standing status.

12. The walking assist device of claim 1, wherein the step cue is a light array or at least one image.

13. The walking assist device of claim 1, wherein the step cue comprises a move-straight forward motion and a turning motion.

14. The walking assist device of claim 1, wherein the walking assist device controls the generation of the step cues by using left and right inconsistent step cues.

15. A method for controlling a step cue of a walking assist device, the method comprising:
generating a detected signal corresponding to a movement of a user;
generating a step length corresponding to the movement of the user;
determining a step status according to the detected signal;
generating a control signal according to the step status and the step length; and
outputting the step cue according to the control signal.

16. The method of claim 15, wherein the detected signal comprises an X-axis acceleration signal, a Y-axis acceleration signal, and a Z-axis acceleration signal; wherein the step status is determined by the Z-axis acceleration signal; and the step length is generated by the X-axis acceleration signal and the Y-axis acceleration signal.

17. The method of claim 16, wherein the step length is compensated by the Z-axis acceleration signal.

18. The method of claim 15, wherein the detected signal comprises a front area detected signal, an inner side detected signal, an outer side detected signal, and a rear area detected signal; wherein the step cue comprises a move-straight forward motion and a turning motion; wherein one of a move-straight forward motion and a turning motion is determined by the inner side detected signal and the outer side detected signal.

19. The method of claim 15, wherein an outputted distance of the step cue is adjusted by a previous calculated step length and a distance of the previous outputted step cue.

20. The method of claim 15, wherein the step cue comprises a move-straight forward motion and a turning motion, and the step cues are generated by using left and right inconsistent step cues.
